(19) United States
(12) Patent Application Publication
(10) Pub. No.: US 2021/0261898 A1
(43) Pub. Date: Aug. 26, 2021

(54) A RECONFIGURABLE BIOLOGICAL COMPUTER BASED ON COUPLED TRAINABLE NEURONAL GATES

(71) Applicant: KONIKU INC., San Rafael, CA (US)

(72) Inventors: Renaud RENAULT, Newark, CA (US); Oshiorenoya E. AGABI, Dublin, CA (US)

(21) Appl. No.: 17/272,226

(22) PCT Filed: Jun. 22, 2018
(86) PCT No.: PCT/US2018/039061

§ 371 Date: Feb. 26, 2021

Related U.S. Application Data
(60) Provisional application No. 62/524,305, filed on Jun. 23, 2017.

(51) Int. Cl. C12M 3/06; G06N 3/06

(52) U.S. Cl. CPC C12M 23/16; G06N 3/061

(57) ABSTRACT

Neuronal gate devices that function as a biological equivalent of AND, OR, and NOT digital logic gates are described. The neuronal gate devices comprise microstructures that support the patterned growth of two or more populations of neurons that are synaptically coupled. Also described are reconfigurable biological computers that comprise two or more coupled neuronal gates and may be trained to process a set of one or more input signals and generate a corresponding set of one or more output signals.

### CROSS-REFERENCE

This application claims the benefit of U.S. Provisional Application No. 62/524,305, filed Jun. 23, 2017, which application is incorporated herein by reference.

### SUMMARY

The present disclosure is directed to creating a biological implementation of the artificial neural networks commonly used in machine learning and artificial intelligence applications. The biological computer can be reconfigured and trained to accomplish different tasks either in an explicit way (training mode 1) or in a goal-oriented way (training mode 2).

The biological computer may comprise: 1) A chip containing at least one but preferably many neuronal gates each comprising at least two but preferably more populations of biological neurons that are synaptically connected; and 2) An interface to record and/or stimulate different neuronal populations in different neuronal gates. This interface can couple distant populations that do not belong to the same neuronal gate, hence creating larger pseudo-networks.

This disclosure provides a neuronal gate device comprising: a) a microstructure comprising at least one input chamber and at least one output chamber, wherein the at least one input chamber is connected with the at least one output chamber through a series of two or more microchannels; and b) at least two populations of neurons, wherein at least one population of neurons resides substantially within the at least one input chamber, and wherein at least one population of neurons resides substantially within the at least one output chamber; wherein the populations of neurons within the at least one input chamber and the at least one output chamber are synaptically connected through the series of two or more microchannels. In some cases, the device comprises a single input chamber connected to a single output chamber and functions as a NOT gate. In some cases, the device comprises two input chambers and a single output chamber and functions as an AND gate. In some cases, the device comprises two input chambers and a single output chamber and functions as an OR gate. In some cases, the device further comprises a modulatory chamber connected with the at least one output chamber, wherein the modulatory chamber comprises a population of neurons that are synaptically connected with those in the output chamber and which function to modulate an output signal of the device. In some cases, the microstructure is fabricated from glass, fused silica, silicon, photoresist, an elastomer, a polymer, or any combination thereof. In some cases, the chambers have a maximum lateral dimension ranging from about 0.1 mm to about 2 mm. In some cases, the chambers have a depth ranging fro [symbol] meters to about 15 micrometers. In some cases, the sy [symbol] between the population of neurons residing substantially within at least one input chamber and the population of neurons residing substantially within at least one output chamber is directional. In some cases, directional synaptic connection is implemented through the use of tapered microchannels to direct axon growth. In some cases, the at least two populations of neurons comprise hippocampal neurons, cortical neurons, striatal neurons, or any combination thereof. In some cases, the population of neurons residing substantially within at least one input chamber or output chamber is a homogeneous population of neurons, glial cells or astrocytes, Schwann cells, olfactory neuron cells, motor neurons, CNS neurons, Cortical neurons or any cells of neural origin. In some cases, the population of neurons residing substantially within at least one input chamber or output chamber is a mixed population of neurons, glial cells or astrocytes, Schwann cells, olfactory neuron cells, motor neurons, CNS neurons, Cortical neurons or any cells of neural origin. In some cases, the population of neurons residing substantially within the modulatory chamber comprises dopaminergic neurons. In some cases, the dopaminergic neurons function to provide a feedback signal to minimize an error signal comprising the difference between an output signal of the device and a desired output signal. In some cases, the synaptic connection between at least one input chamber and at least one output chamber is increased or decreased using a training regimen of applying paired stimuli to the neurons residing substantially within the input and output chambers in defined temporal patterns. In some cases, a time delay between an input stimulus and an output stimulus of the paired stimuli used for training ranges from about −50 milliseconds to about +50 milliseconds. In some cases, the paired stimuli are applied repetitively, and wherein a number of repetitions used for training ranges from about 10 to about 1,000. In some cases, a frequency of repetition ranges from about 0.1 Hz to about 100 Hz. In some cases, the paired stimuli comprise optical signals, electrical signals, or any combination thereof. In some cases, the device further comprising at least one integrated input stimulus probe and at least one integrated output stimulus probe. In some cases, the integrated input and output stimulus probes comprise a light-emitting diode (LED), an electrode, or any combination thereof. In some cases, the at least one integrated output signal recording probe. In some cases, the at least one integrated output signal recording probe comprises a photodiode, and avalanche photodiode, a CMOS image sensor, an electrode, or any combination thereof.

Described herein is a reconfigurable biological computer comprising: a) a microchip comprising two or more coupled neuronal gate devices, wherein each neuronal gate device comprises: i) a microstructure comprising at least one input chamber and at least one output chamber, wherein the at least one input chamber is connected with the at least one output chamber through a series of two or more microchannels; and ii) at least two populations of neurons, wherein at least one population of neurons resides substantially within the at least one inp [symbol] wherein at least one population of neurons resides su [symbol] at least one output chamber; wherein the populations of neurons within the at least one input chamber and the at least one output chamber are synaptically connected though the series of two or more microchannels; and b) an interface device configured to stimulate at least one population of neurons within the microchip, record an output signal from at least one population of neurons within the microchip, or any combination thereof. In some cases, at least one of the two or more coupled neuronal gate devices further comprises a modulatory chamber connected with the at least one output chamber, wherein the modulatory chamber comprises a population of neurons that are synaptically connected with those in the output chamber and which function to modulate an output signal of the device. In some cases, the synaptic connections between the two or more coupled neuronal gate devices are configured to emulate a neural network functionality that maps a set of one or more input signals to a set of one or more output signals. In some cases, a set of one or more signals derived from a cell-based sensor array are used as input signals for the microchip, and the neural network functionality is used to identify a specific volatile compound or mixture of compounds detected by the cell-based sensor array. In some cases, the synaptic connections between the populations of neurons within at least one input chamber and at least one output chamber of the coupled neuronal gate devices have been reinforced through a training process comprising applying one or more sets of paired input and output stimuli in defined temporal patterns. In some cases, the a time delay between an input stimulus and an output stimulus of the paired stimuli used for training ranges from about −50 milliseconds to about +50 milliseconds. In some cases, the paired stimuli are applied repetitively, and wherein a number of repetitions used for training ranges from about 10 to about 1,000. In some cases, a frequency of repetition ranges from about 0.1 Hz to about 100 Hz. In some cases, the paired stimuli comprise optical signals, electrical signals, or any combination thereof. In some cases, the interface device applies a stimulus signal or records an output signal that comprises an optical signal, an electrical signal, or any combination thereof. In some cases, the microstructure is fabricated from glass, fused silica, silicon, photoresist, an elastomer, a polymer, or any combination thereof. In some cases, the chambers of the microstructure have a maximum lateral dimension ranging from about 0.1 mm to about 2 mm. In some cases, the chambers of the microstructure have a depth ranging from about 2 micrometers to about 15 micrometers. In some cases, the synaptic connection between the population of neurons residing substantially within at least one input chamber and the population of neurons residing substantially within at least one output chamber is directional. In some cases, the directional synaptic connection is implemented through the use of tapered microchannels to direct axon growth. In some cases, the at least two populations of neurons comprise hippocampal neurons, cortical neurons, striatal neurons, or any combination thereof. In some cases, the population of neurons residing substantially within at least one input chamber or out [symbol] a homogeneous population of neurons. In some cases, [symbol] neurons residing substantially within at least one input chamber or output chamber is a mixed population of neurons. In some cases, the population of neurons residing substantially within the modulatory chamber comprises dopaminergic neurons. In some cases, the dopaminergic neurons function to provide a feedback signal to minimize an error signal comprising the difference between an output signal of a neuronal gate device and a desired output signal. In some cases, the two or more coupled neuronal gate devices further comprise at least one integrated input stimulus probe and at least one integrated output stimulus probe. In some cases, the integrated input and output stimulus probes comprise a light-emitting diode (LED), an electrode, or any combination thereof. In some cases, the two or more coupled neuronal gate devices further comprise at least one integrated output signal recording probe. In some cases, the at least one integrated output signal recording probe comprises a photodiode, and avalanche photodiode, a CMOS image sensor, an electrode, or any combination thereof. In some cases, a computing task performed by the computer can be reconfigured through a training regime comprising repetitive delivery of pairwise input and output stimuli to the populations of neurons residing substantially within at least one input chamber and at least one output chamber.

Described herein is a method of making a neuronal gate device comprising, assembling: a microstructure comprising at least one input chamber and at least one output chamber wherein the at least one input chamber is connected with the at least one output chamber through a series of two or more microchannels; and placing at least two populations of neurons, wherein at least one population of neurons is placed substantially within the at least one input chamber, and wherein at least one population of neurons is placed substantially within the at least one output chamber; and sypantically connecting the populations of neurons within the at least one input chamber and the at least one output chamber, through the series of two or more microchannels. A method of making a reconfigurable computer comprising, assembling: a) a microchip comprising two or more coupled neuronal gate devices, wherein each neuronal gate device comprises: i) a microstructure comprising at least one input chamber and at least one output chamber, wherein the at least one input chamber is connected with the at least one output chamber through a series of two or more microchannels; and ii) at least two populations of neurons, wherein at least one population of neurons resides substantially within the at least one input chamber, and wherein at least one population of neurons resides substantially within the at least one output chamber; wherein the populations of neurons within the at least one input chamber and the at least one output chamber are synaptically connected though the series of two or more microchannels; and b) an interface device configured to stimulate at least one population of neurons within the microchip, record an output signal from at least one population of neurons within the microchip, or any combination thereof.

[symbol] Werein is a method of using reconfigurable computer as [symbol] disclosure, in a system requiring high computational power. Described herein is a method of using reconfigurable computer as provided in this disclosure, in an application requiring analysis of a data-set derived from an unstructured variable. Described herein is a method of using reconfigurable computer as provided in this disclosure, in a robotics application; in an application comprising experimental neuroscience; in memory modeling; in drug discovery; in making synthetic organisms; in making living machines; in edge computing, or any combinations thereof.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A shows a schematic representation of a neuronal gate microstructure comprising two chambers for containing populations of neurons that are synaptically connected through a series of microchannels that connect the two chambers.

FIG. 1B shows a fluorescence microscopy image of the neuronal gate microstructure of FIG. 1A showing the two populations of neurons two weeks after seeding of the neurons.

FIG. 1C shows a schematic representation of a neuronal gate microstructure comprising three chambers, each containing a populations of neurons, with two functioning as inputs and one functioning as an output.

FIG. 1D shows a fluorescence microscopy image of the neuronal gate microstructure of FIG. 1C showing the three populations of neurons two weeks after seeding of the neurons.

FIG. 2 shows a schematic representation of a protocol for training a neuronal gate in an explicit way (training mode 1).

FIG. 3 shows a schematic representation of a trainable neuronal gate with 2 inputs, 1 output and 1 modulatory compartment. This additional compartment may be seeded with dopaminergic neurons and may innervate the output population of neurons to modulate training.

[symbol] ws a diagram indicating coupling of 3 neuronal gates a [symbol] work.

FIG. 5 shows a schematic illustration of an artificial neural network (ANN).

FIG. 6 shows a schematic illustration of a deep learning neural network (DNN).

FIG. 7 provides a schematic illustration of the functionality of a node within a layer of an artificial neural network or deep learning neural network.

FIG. 8 provides a schematic illustration of an ordered seeding of neurons in the biocompatible polymer and connecting the paths in straight line or otherwise.

FIG. 9 provides a dis-ordered seeding of neurons into polymer and connecting the paths in a straight line or otherwise.

FIG. 10 provides a schematic illustration of circuits in the 3D space of hydrogel with connecting paths in straight lines or otherwise and 3D electrodes on the bottom or side.

FIG. 11 provides a schematic illustration of connected circuits with reader neurons and perfusion paths which doubly serve as waveguides.

FIG. 12 provides a device with a re-aggregate framework added to the system.

FIG. 13 provides a schematic illustration of a stimulation array at the top or bottom integrated with the base.

FIG. 14 provides a schematic illustration of a device realization with paths built in a biocompatible polymer.

FIG. 15 provides a demonstration of the system with neurons and paths in a 3D biocompatible polymer.

### DETAILED DESCRIPTION

Disclosed herein is a biological computer composed of genetically modified neurons. The genetically modified neurons, also referred to as synthetic biological neurons, may be able to live outside of the naturally evolved state while maintaining their information processing abilities, within a core system. The genetically modified neurons or synthetic biological neurons can have a biological genetic code which prevents them from freezing in extreme environments; may be able to tolerate extreme conditions, such as radiation and extreme pH changes by borrowing genetic codes from extremophiles. In one example is provided a system where the neural architecture is explicitly defined. In some examples, explicit or other methods of training may be employed to train a neuronal gate as described herein.

Disclosed herein are neuronal gate devices that provide a biological equivalent to the digital logic gates that provide the functional building blocks from which digital electronic circuits and microprocessors are constructed. Electronic digital logic gates perform the logical operations of AND, OR and NOT on binary numbers, i.e., numbers expressed in the base-2 or bin [symbol] tem which may be represented using strings of only tv [symbol] ly 0 (zero) and 1 (one). Unlike electronic digital logic gates, the disclosed neuronal gate devices may, in some cases, operate in input signals that are not binary inputs (i.e., non-binary, real-valued input values), and may output non-binary, real-valued output values.

The disclosed neuronal gate devices may comprise a) a microstructure comprising at least one input chamber and at least one output chamber, wherein the at least one input chamber is connected with the at least one output chamber through a series of two or more microchannels; and b) at least two populations of neurons, wherein at least one population of neurons resides substantially within the at least one input chamber and at least one population of neurons resides substantially within the at least one output chamber; and wherein the populations of neurons within the at least one input chamber and the at least one output chamber are synaptically connected though the series of two or more microchannels. Other cell types within the chambers may include, glial cells or astrocytes, Schwann cells, olfactory neuron cells, motor neurons, CNS neurons, cortical neurons or any cells of neural origin. They may also include dividing or non-dividing HEK cells. In some cases, the neuronal gate device may further comprise at least one modulatory chamber connected with the at least one output chamber, wherein the modulatory chamber comprises a population of neurons that are synaptically connected with those in the output chamber and which function to modulate an output signal of the device. In some cases, the synaptic connection between at least one input chamber and at least one output chamber is modulated, e.g., the degree of connection may be increased or decreased, using a training regimen comprising application of paired stimuli to the neurons residing within the input and output chambers, where the paired stimuli are applied in defined temporal patterns. In addition, synthetic cells, such as, cells made from scratch with from a lipid bilayer and reconstituted or extensively modified cells which may even incorporate silicon elements within the cell or grown silicon elements within them to make them operate at faster cycles.

Also disclosed herein are reconfigurable biological processors or computers comprising two or more coupled neuronal gate devices, and methods for training said processors or computers to perform specific tasks. For example, in some cases, the reconfigurable biological processor or computer may function as a biological equivalent of an artificial neural network, and may be trained to map a set of one or more inputs (i.e., input signals, input data sets or input data vectors) to a set of one or more outputs (i.e., output signals, output data sets, or output data vectors).

The disclosed reconfigurable biological processors or computers may comprise: a) a microchip comprising two or more coupled neuronal gate devices, wherein each neuronal gate device comprises: i) a microstructure comprising at least one input chamber and at least one output chamber, wherein the at least one input chamber is connected with the at least one output cha [symbol] series of two or more microchannels; and ii) at least t [symbol] neurons, wherein at least one population of neurons resides substantially within the at least one input chamber, and wherein at least one population of neurons resides substantially within the at least one output chamber; wherein the populations of neurons within the at least one input chamber and the at least one output chamber are synaptically connected though the series of two or more microchannels; and b) an interface device configured to stimulate at least one population of neurons within the microchip, record an output signal from at least one population of neurons within the microchip, or any combination thereof. In some cases, at least one of the two or more coupled neuronal gate devices further comprises a modulatory chamber connected with the at least one output chamber, wherein the modulatory chamber comprises a population of neurons that are synaptically connected with those in the output chamber and which function to modulate an output signal of the device. In some cases, the disclosed biological processors or computers may be trained to perform a specified task through a training process comprising applying one or more sets of paired input and output stimuli in defined temporal patterns.

The disclosure will be described using a terminology understandable by someone knowledgeable in the field of microfabrication, microfluidics, neuroscience, optics and electronics.

As used in this specification and the appended claims, the singular forms “a”, “an”, and “the” include plural references unless the context clearly dictates otherwise. Any reference to “or” herein is intended to encompass “and/or” unless otherwise stated.

As used herein, the term ‘about’ a number refers to that number plus or minus 10% of that number. The term ‘about’ when used in the context of a range refers to that range minus 10% of its lowest value and plus 10% of its greatest value.

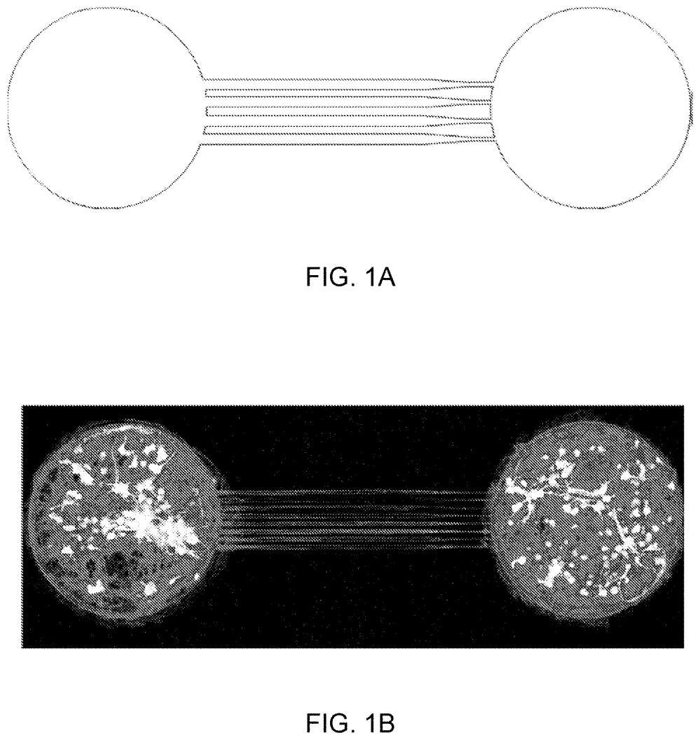

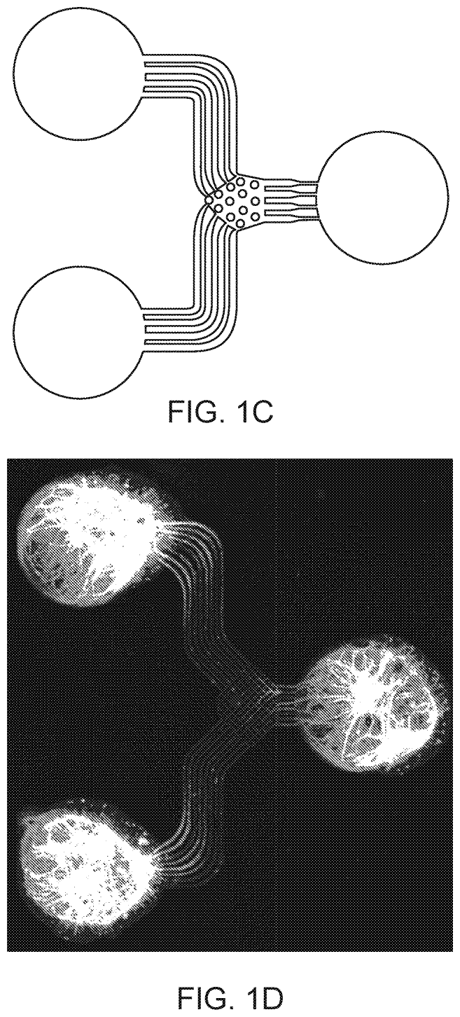

Microstructures and microchips: In some cases, a neuronal gate device of the present disclosure may comprise a microstructure containing live neurons. In some cases, the microstructures for a biological processor comprising two or more coupled neuronal gate devices may be integrated within a single microchip containing live neurons. In some cases, an apparatus or system of the present disclosure may comprise one or more biological processors that each comprise a microchip containing live neurons. In some cases, the microstructure and/or microchip may comprise a planar substrate (such as glass, borosilicate, fused-silica, silicon, photoresist, an elastomer, a polymer, or any combination thereof), endowed with two or more microwells (or chambers) in which biological neurons may be seeded, and a series of microchannels (or microtunnels) connecting, for example, an input chamber and an output chamber, in which axons can grow from one population of neurons to the other (FIG. 1A).

The different microstructures within a microchip (e.g., microwells, chambers, and/or microchannels) can be fabricated using any of a variety of techniques known to those of skill in the [symbol] technique may be dependent on the choice of material [symbol] Examples of suitable fabrication techniques and materials include, but are not limited to, direct 3D printing on the substrate using stereo-photolithography or bi-photon 3D lithography of biocompatible photoresists like SU-8, or micromolding from a negative master mold (made of e.g. SU-8 on silicon) in polydimethylsiloxane (PDMS), followed by bonding to the substrate using plasma surface activation.

In some cases, the number of microwells or chambers per microstructure or microchip may be at least 1, 2, 3, 4, 5, 6, 7, 8, 9, 10, or more. In some cases the number of microchannels connecting any two microwells or chambers within the microstructure or microchip may be at least 1, 2, 3, 4, 5, 6, 7, 8, 9, 10, or more.

The maximum lateral dimension (e.g., within the plane of substrate within which the chambers are fabricated) of the microwells or chambers within which the populations of neurons substantially reside may range from about 0.1 millimeters (mm) to about 2 mm in diameter, and the microwells or chambers may be connected by a series of microchannels with internal dimensions ranging from about 2 micrometers (um) to about 15 um in width or diameter. In some cases, the maximum lateral dimension of the microwells or chambers may be at least 0.1 mm, at least 0.2 mm, at least 0.3 mm, at least 0.4 mm, at least 0.5 mm, at least 1.0 mm, at least 1.5 mm, or at least 2 mm. In some cases, the maximum lateral dimension of the microwells or chambers may be at most 2 mm, at most 1.5 mm, at most 1.0 mm, at most 0.5 mm, at most 0.4 mm, at most 0.3 mm, at most 0.2 mm, or at most 0.1 mm. Any of the lower and upper values described in this paragraph may be combined to form a range included within the present disclosure, for example, the maximum lateral dimension of the microwells or chambers may range from about 0.4 mm to about 1.5 mm. Those of skill in the art will recognize that the maximum lateral dimension of the microwells or chambers may have any value within this range, e.g., about 1.25 mm. In some cases, the internal dimensions of the microchannels connecting the microwells or chambers may be at least 2 um, at least 4 um, at least 6 um, at least 8 um, at least 10 um, or at least 15 um in width or diameter. In some cases, the internal dimensions of the microchannels connecting the microwells or chambers may be at most 15 um, at most 10 um, at most 8 um, at most 6 um, at most 4 um, or at most 2 um. Any of the lower and upper values described in this paragraph may be combined to form a range included within the present disclosure, for example, the internal dimensions of the microchannels connecting the microwells or chambers may range from about 4 um to about 10 um in width or diameter. Those of skill in the art will recognize that the internal dimensions of the microchannels connecting the microwells or chambers may have any value within this range, e.g., about 8.5 um in width or diameter.

[symbol] es, it may be desirable to have directional connectivity [symbol] ons of neurons. In some such cases, microchannels can take specific shapes (e.g., tapered microchannels (as illustrated in FIG. 1A), lateral arches, etc.) to direct axonal growth. Lateral arches, also referred to as tunnels, can be two dimensional or three dimensional paths which can control the direction of growth of the neuron growth cone. A biocompatible polymer or other materials may be employed atop the planar substrate, such that tunnels may be built within this biocompatible polymer.

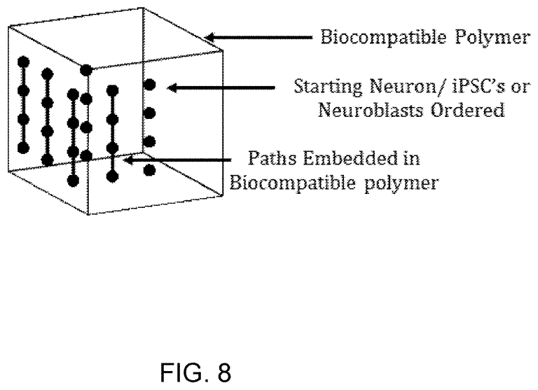

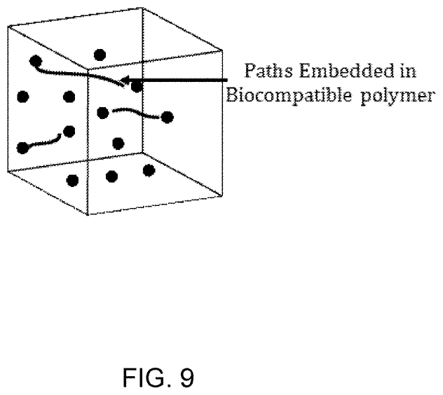

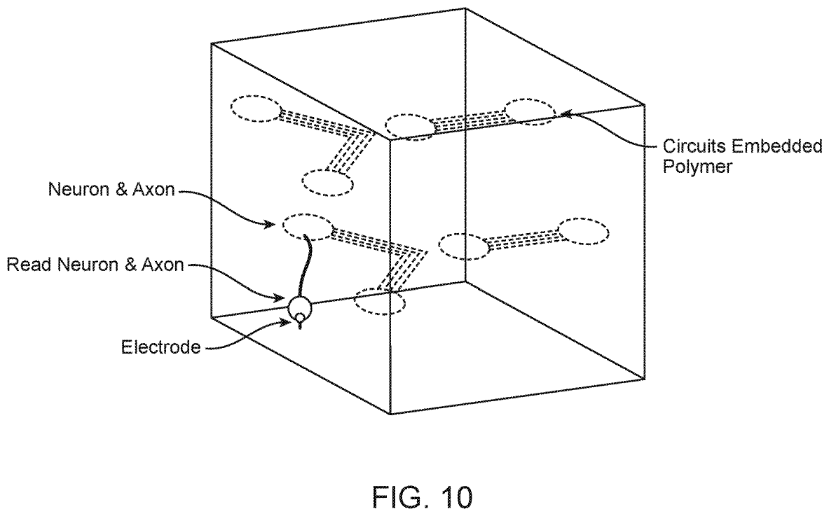

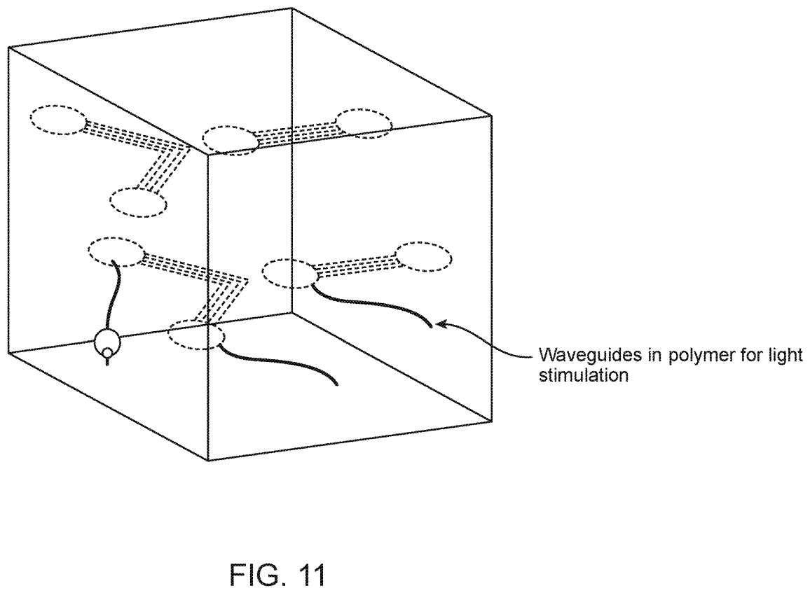

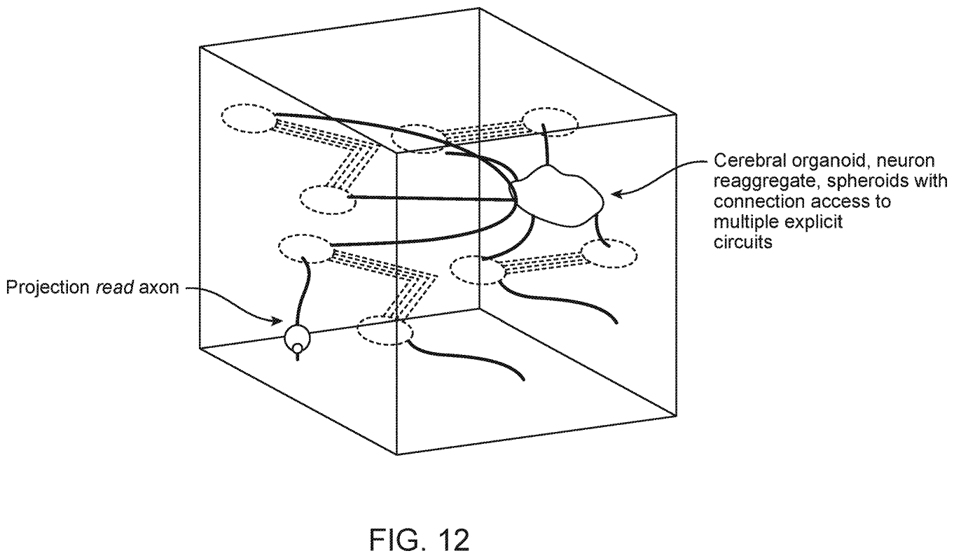

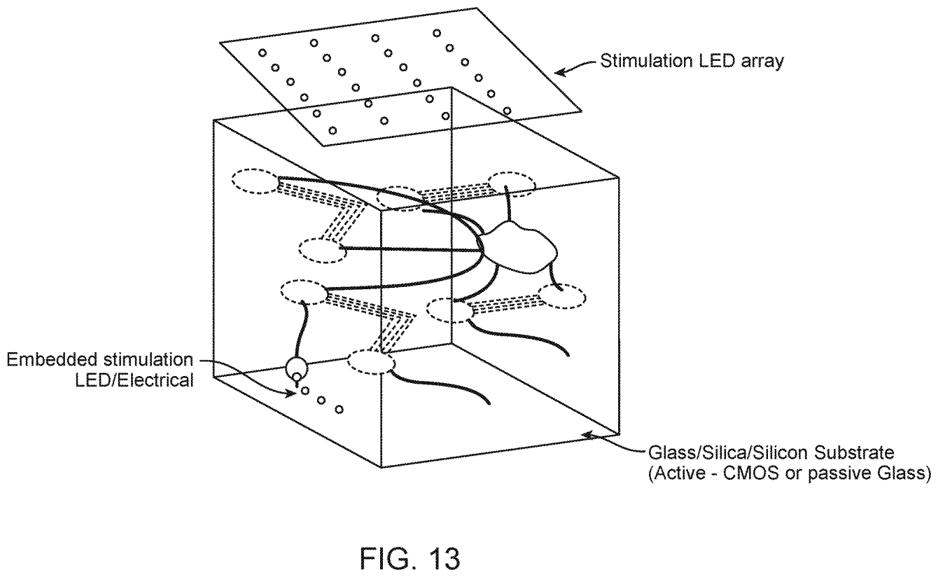

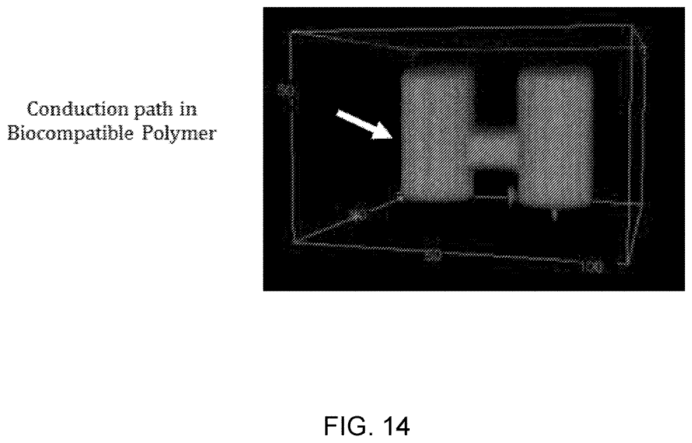

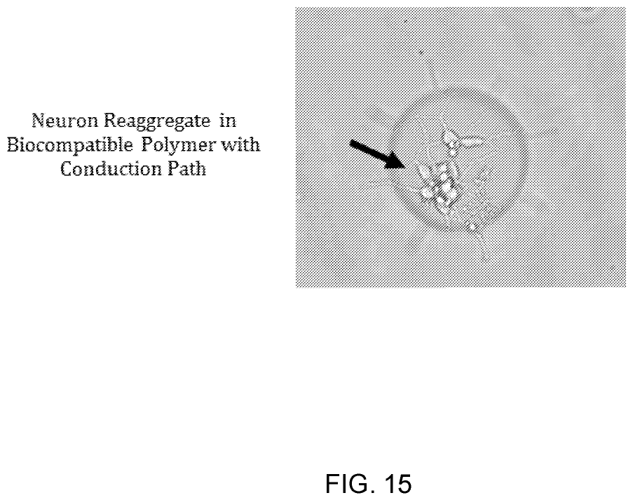

Seeding of neurons into microwells or chambers: Dissociated neurons, typically derived from rodent primary tissues or differentiated mammalian stem cells, may be seeded in the different microwells or chambers. In some cases, the different microwells or chambers may be coated (such as prior to seeding) with an adhesive molecule such as poly-lysine, fibronectin to promote adhesion of the neurons to the surfaces of the microwells or chambers. The neurons may be incubated at an appropriate incubation temperature and allowed to grow for from about 2 weeks to about 3 weeks in an appropriate culture medium. During this time, the neurons may establish functional synapses with each other, within different populations of neurons, between different populations of neurons, or any combination thereof (FIG. 1B). Dissociated neurons which may vary in type, morphology, protein expression, genetic makeup or other phenotypes may be seeded in an ordered or unordered fashion within the hydrogel or other biocompatible polymer. The perfusion of this system may be accomplished with secondary wicking polymers embedded within the hydrogel or by explicitly defining perfusion channels within the biocompatible polymer super structure. Post seeding of these neurons and other supporting cell types in the hydrogel, a multiphoton microscope can be used to direct the growth of circuits within this substrate. The use of multiphoton lithography to direct the growth of circuits within this system may be sequential or parallel, for example, using a galvanometric directed circuit imprinting, holographic imprinting, 3D micro printing, or even metallic micro printing of the conducting sections with electroless deposition techniques. Such methods are especially useful for getting access to the non-read neurons with biocompatible metals such as gold, platinum and similar. The neurons grown within the upper layers of the hydrogel or other biocompatible polymer may not necessarily be the “read” neurons. The neurons on the bottom layer or side layers can be the “read” neurons. Connection between neurons in the super structure and the read neurons are realized by directing the growth of neurons upwards, sideways or downwards depending on where the read neurons are grown. The read neurons may be grown directly on 3D electrodes which project directly to the circuits in the middle of biocompatible polymer. The read neurons can also be circuits in their own right. A schematic illustration of an ordered seeding of neurons in the biocompatible polymer and connecting the paths in straight line or otherwise in shown in FIG. 8. FIG. 9 shows an example of a dis-ordered seeding of neurons into polymer and co [symbol] as in a straight line or otherwise. FIG. 10 shows an ex [symbol] the 3D space of hydrogel with connecting paths in straight lines or otherwise and 3D electrodes on the bottom or side. FIG. 11 shows an example of connected circuits with reader neurons and perfusion paths which doubly serve as waveguides. FIG. 12 shows a device with a re-aggregate framework added to the system. FIG. 13 shows a schematic illustration of a stimulation array at the top or bottom integrated with the base. FIG. 14 shows a schematic illustration of a device realization with paths built in a biocompatible polymer. FIG. 15 provides an exemplary demonstration of the system with neurons and paths in a 3D biocompatible polymer.

Training to induce synaptic plasticity: In some cases, the synaptic connectivity between different populations of neurons within a neuronal gate device or reconfigurable biological processor or computer, may be modified through the use of a training regimen. For example, the training regimen may comprise inducing changes in the strength of synapses between an input population (i.e., a population of neurons residing substantially within an input microwell or chamber) and an output population (i.e., a population of neurons residing substantially within an output microwell or chamber). The training may thus induce synaptic plasticity—a biological process by which specific patterns of synaptic activity result in changes in synaptic strength. Synaptic plasticity in the brain is thought to contribute to learning and memory. Both pre-synaptic and post-synaptic mechanisms can contribute to the expression of synaptic plasticity.

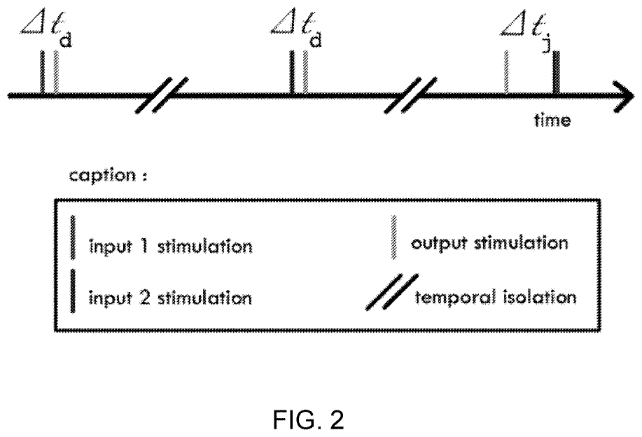

In some cases, a training regimen comprising repeated paired stimulations of input populations of neurons and output populations of neurons in defined temporal patterns may increase or decrease a connectivity between the input and output populations. This may be called spike timing dependent plasticity (STDP). For a neuronal gate comprising one input and one output, a relevant parameter for this induction protocol may be: (i) a delay between the input and output stimulation (typically from about −50 millisecond (ms) to about +50 ms); (ii) a number of repetitions (typically from about 10 to about 1000); (iii) a frequency of repetition (typically from about 0.1 Hertz (Hz) to about 100 Hz); (iv) a strength of stimuli, or (v) any combination thereof. For a device with two inputs and one output (FIG. 1C-D), the parameter space for the induction protocol may be even larger, but due to symmetry considerations it may simply be reduced to two disjoint paired stimulations (one for each of the two input-output pairs) with the same delay and a joint paired stimulation (where both inputs may be stimulated simultaneously) with a different delay (FIG. 2). This type of training (training mode 1) may be completely supervised and explicit, as it may require each link to be tuned individually to match a desired connectivity.

In some cases, the time delay between paired input and output stimuli used for training may be at least −50 ms, at least −40 ms, at least −30 ms, at least −20 ms, at least −10 ms, at least 0 ms, at least +10 ms, at least +20 ms, at least +30 ms, at least +40 ms, or at least +50 ms. In some cases, the time delay between paired input and output stimuli used for training may be at most +5 [symbol] 10 ms, at most +30 ms, at most +20 ms, at most +10 m [symbol] most −10 ms, at most −20 ms, at most −30 ms, at most −40 ms, or at most −50 ms. Any of the lower and upper values described in this paragraph may be combined to form a range included within the present disclosure, for example, the time delay between paired input and output stimuli used for training may range from about −20 ms to about +40 ms. Those of skill in the art will recognize that the time delay between paired input and output stimuli used for training may have any value within this range, e.g., about +12.5 ms.

In some cases, the number of repetitions of paired input and output stimuli used for training may be at least 10, at least 20, at least 30, at least 40, at least 50, at least 60, at least 70, at least 80, at least 90, at least 100, at least 200, at least 300, at least 400, at least 500, at least 600, at least 700, at least 800, at least 900, or at least 1,000. In some cases, the number of repetitions of paired input and output stimuli used for training may be at most 1,000, at most 900, at most 800, at most 700, at most 600, at most 500, at most 400, at most 300, at most 200, at most 100, at most 90, at most 80, at most 70, at most 60, at most 50, at most 40, at most 30, at most 20, or at most 10. Any of the lower and upper values described in this paragraph may be combined to form a range included within the present disclosure, for example, the number of repetitions of paired input and output stimuli used for training may range from about 30 to about 600. Those of skill in the art will recognize that the number of repetitions of paired input and output stimuli used for training may have any value within this range, e.g., about 22.

In some cases, the frequency of repetition of paired input and output stimuli used for training may be at least 0.1 Hz, at least 0.5 Hz, at least 1 Hz, at least 5 Hz, at least 10 Hz, at least 50 Hz, or at least 100 Hz. In some cases, the frequency of repetition of paired input and output stimuli used for training may be at most 100 Hz, at most 100 Hz, at most 50 Hz, at most 10 Hz, at most 5 Hz, at most 1 Hz, at most 0.1 Hz. Any of the lower and upper values described in this paragraph may be combined to form a range included within the present disclosure, for example, the frequency of repetition of paired input and output stimuli used for training may range from about 1 Hz to about 50 Hz. Those of skill in the art will recognize that the frequency of repetition of paired input and output stimuli used for training may have any value within this range, e.g., about 12 Hz.

In some cases, the strength of the paired input and output stimuli used for training may vary independently of each other and may range from about 0% to about 100% of a maximum stimulus strength. In some cases, the strength of the paired input and/or output stimuli may be at least 0%, at least 10%, at least 20%, at least 30%, at least 40%, at least 50%, at least 60%, at least 70%, at least 80%, at least 90%, or at least 100% of a maximum stimulus strength. In some cases, the strength of the paired input and/or output stimuli may be at most 100%, at most 90%, at most 80%, at most 70%, at most 60%, at most 50%, at most 40%, at most 30%, at most 20%, at [symbol] most 0% of a maximum stimulus strength. Any of the [symbol] values described in this paragraph may be combined to form a range included within the present disclosure, for example, the strength of the paired input and/or output stimuli may range from about 10% to about 80% of a maximum stimulus strength. Those of skill in the art will recognize that the strength of the paired input and/or output stimuli may have any value within this range, e.g., about 65% of a maximum stimulus strength.

Any of a variety of stimulus signals known to those of skill in the art may be used as input and/or output stimuli for training the neuronal gate devices (and reconfigurable biological computers) disclosed herein. Input and/or output stimuli of various types may be delivered to one or more populations of neurons within a device using integrated stimulus probes and/or externally positioned stimulus probes. Examples include, but are not limited to, electrical stimuli (delivered by means of integrated or externally positioned electrodes), optical stimuli (e.g., optogenetic stimuli in the event that one or more populations of neurons within the device have been genetically engineered to express light-sensitive ion channels, and the optical stimuli are delivered by means of integrated or externally positioned light emitting diodes (LEDs), diode lasers, optical fiber-based light sources, etc.), chemical stimuli (delivered by means of integrated microchannels or externally positioned micropipetting devices), or any combination thereof.

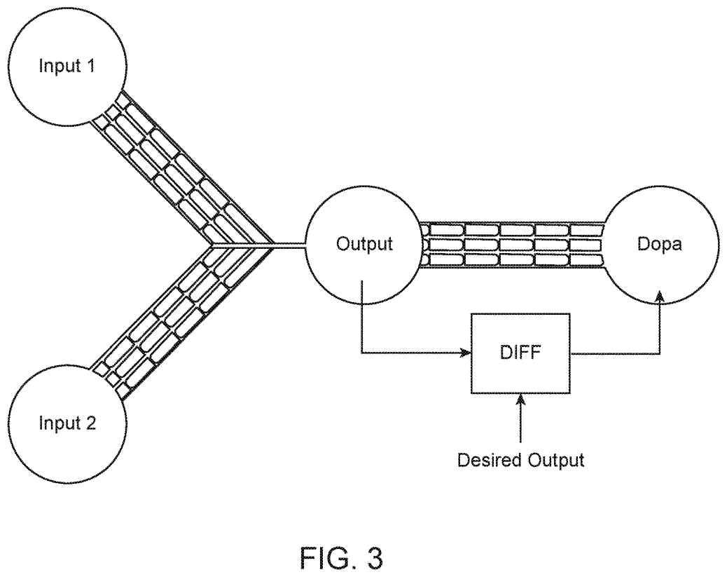

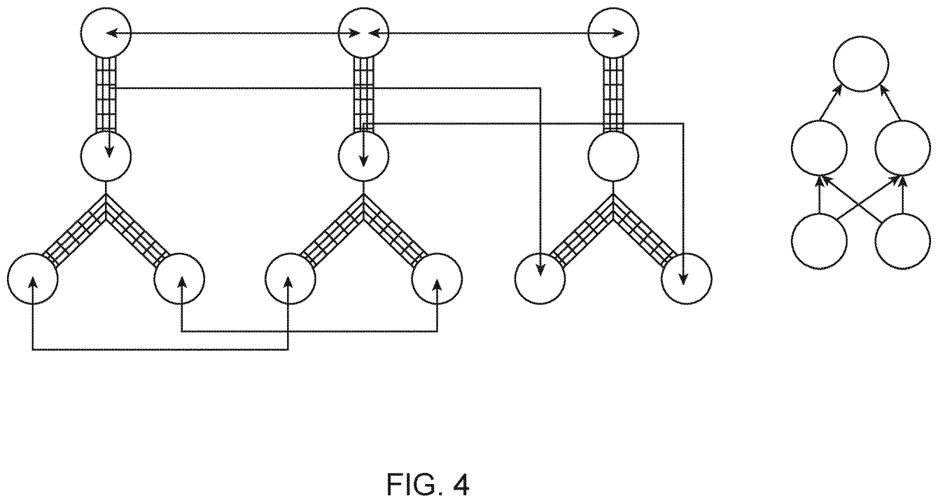

Training and modulation of output signals: Evidence suggests that a neuromodulator like dopamine can facilitate learning so that learning may be effective at lesser number of stimulation cycles during induction. In some cases, learning may be possible without any induction. Therefore, additional populations of neurons (such as dopaminergic neurons residing substantially within one or more modulatory chambers within the device) may be integrated into neuronal gate devices to modulate learning (FIG. 3). In some cases, a dopamine feedback circuit may be utilized to communicate an error signal to a neuronal gate—similar to an in vivo event—where dopaminergic activity may code for an error on a reward prediction, or function to minimize an error signal comprising the difference between an output signal of the device and a desired output signal. In some cases, if an output activity or signal is less than a desired output activity or signal, a modulatory population of neurons may be stimulated. In some cases, if an output activity or signal is greater than a desired output activity or signal, a modulatory population of neurons may be inhibited. This type of training (training mode 2) may be different from training mode 1, as it may consider an output activity or signal and may not consider each synaptic connection explicitly. For example it may not matter which synaptic connections are reinforced as long as it reduces the output error.

Types of neurons: Neuronal populations residing within the disclosed devices can be of different types such as input, output, or modulatory populations. Input, output, modulatory po [symbol] combination thereof may be seeded with one or more [symbol] neurons. In some cases, an input or output population may comprise a hippocampal neuron, a cortical neuron, a striatal neuron, or any combination thereof. In some cases, a modulatory population may comprise dopaminergic neurons.

Reconfigurable biological processors or computers: Disclosed herein are reconfigurable biological processors (or computers) comprising two or more coupled neuronal gate devices. In some cases the two or more coupled neuronal gate devices are coupled through synaptic connections between the output(s) of one or more neuronal gates and the input(s) of one or more neuronal gates. In some cases the two or more coupled neuronal gate devices are coupled through electrical connections (e.g., through integrated electrical contacts or electrodes) between the output(s) of one or more neuronal gates and the input(s) of one or more neuronal gates (e.g., see FIG. 4). The system can be akin to a bridge arch on its side, a sector of circle or a variation thereof, as shown in FIG. 4. In some cases, two unconnected neuronal populations with a device may be operatively coupled, such as by delivering one or more optogenetic stimulations in one neuronal population based on an activity of a second neuronal population, thereby combining several neuronal gates into a larger network.

A configuration file may be loaded into a biological processor or computer device to specify a particular network to be implemented. A network may be trained, and after training it may be exploited for any of a variety of tasks known to those of skill in the art. For example, in some cases, the reconfigurable biological processor or computer functions as a biological equivalent to an artificial neural network, and may be trained to map a set of input signals (or input data, input data vectors, etc.) to an output (e.g., an output signal, an output data set, or an output data vector). In some cases, for example, a set of one or more signals derived from a cell-based sensor array are used as input signals for the biological processor, and the neural network functionality is used to identify a specific volatile compound or mixture of compounds detected by the cell-based sensor array. In general, the disclosed reconfigurable biological processors or computers may comprise: a) a microchip comprising two or more coupled neuronal gate devices, wherein each neuronal gate device comprises: i) a microstructure comprising at least one input chamber and at least one output chamber, wherein the at least one input chamber is connected with the at least one output chamber through a series of two or more microchannels; and ii) at least two populations of neurons, wherein at least one population of neurons resides substantially within the at least one input chamber, and wherein at least one population of neurons resides substantially within the at least one output chamber; wherein the populations of neurons within the at least one input chamber and the at least one output chamber are synaptically connected though the series of two or more microchannels; and b) an interface device configured to stimulate at least one population of neurons within the microchip, record an output signal from at lea [symbol] n of neurons within the microchip, or any combination [symbol] cal computer, or a reconfigurable computer, as described herein can be very powerful for systems which require high computational power, low power and massive space constraints. In some examples, human or humanoid sized applications can be contemplated. Applications which require integration of high dimensional data sets, e.g., data sets which might arise from unstructured real-world variables, such as sound, light, smell, taste and many other sources. The devices described herein can be suited for robotics, industrial sensing, diagnostics, human perceptome prediction, experimental neuroscience, memory modelling, drug discovery, making synthetic organisms, living machines, edge computing, and other applications.

In some cases, the disclosed reconfigurable biological processors or computers may comprise at least 2, 3, 4, 5, 6, 7, 8, 9, 10, 20, 30 60, 70, 80, 90, 100, 200, 300, 400, 500, 600, 700, 800, 900, 1,000 or more coupled neuronal gate devices.

As noted above, in some cases, the reconfigurable biological processor or computer may comprise a device configured as an interface to read and/or write information into the microchip. The chip reader may be configured to (i) accommodate at least one chip, (ii) stimulate different neuronal populations, (iii) read an activity of one or more different populations in different neuronal gates individually, or (iv) any combination thereof.

Recording responses of neuronal populations may be done optically, electrically, or by using a combination thereof using integrated and/or externally positioned signal recording probes. Optical readout may be performed, for example, by measuring a calcium flux of a neuron loaded with a voltage sensitive fluorescent dye using integrated and/or externally positioned photodiodes, avalanche photodiodes, CMOS image sensors, or any combination thereof. Electrical readout may necessitate (i) an extracellular electrode to be embedded in a substrate under a neuronal population, (ii) conductive tracks connecting an electrode to an amplifier, (iii) an acquisition system to extract a spiking activity of neurons of a neuronal population growing on the conductive tracks, or (iv) any combination thereof. In some cases, electrical readout may be performed using one or more externally positioned electrodes (i.e., not directly integrated with the microchip).

Stimulation of neuronal populations can be done using an optical method, an electrical method, or a combination thereof. An electrical method may comprise, for example, applying one or more biphasic short voltage pulses on different electrodes to elicit one or more spikes in the neurons associated with or contacting them. In some cases, the stimulation electrodes (or stimulation probes) may be integrated with the microchip device. In some cases the stimulation electrodes may be externally positioned. An optical method may comprise, for example, the use of optogenetics in which one or more populations of neurons within the device have been genetically engineered to express light-sensitive ion channels. One or more flashes of light may be [symbol] etically modified, light-sensitive neurons to stimulate [symbol] es, the one or more flashes of light may be provided by one or more integrated light emitting diodes (LEDs) or other integrated light stimulus probes. In some cases, the one or more flashes of light may be provided by one or more LEDs or other light stimulus probes that are not directly integrated with the device but are externally positioned.

In some cases, the stimulation method may comprise the use of chemical stimuli, as noted above in the discussion of training methods.

In some cases, recording and stimulation methods are both optical methods. In some cases, recording and stimulation methods are both electrical methods. In some cases, recording and stimulation methods are not the same. For example, a recording method may be an optical method and a stimulation method may be an electrical method. A recording method may be an electrical method and a stimulation method may be an optical method. Use of recording and stimulation methods that are different may reduce cross-talk, noise, and artifacts in a signal. In some cases, an interface may utilize one or more extracellular electrodes to record spiking activity and optogenetics to stimulate one or more neurons. In some cases, one or more electrodes may be dedicated to each of one or more neuronal populations to obtain an accurate estimation of neuronal electrical activity within each population, while optical fibers coupled to a suitable detector (e.g., a photodiode, avalanche photodiode, a CMOS imaging sensor, a photomultiplier, etc.) may be optically aligned with one or more populations of neurons from one side of the device and light emitting diodes (LEDs) aligned with each of the one or more populations of neurons on an opposite side of the device allow one to optically stimulate and/or record activity for each population of neurons individually.

### EXAMPLES

These examples are provided for illustrative purposes only and not to limit the scope of the claims provided herein.

### Example 1—Neuronal Gate Devices that Function as Digital Logic Gates

FIG. 1A provides a non-limiting example of a neuronal gate microstructure designed to support the growth of two populations of neurons (FIG. 1B), each population of neurons residing substantially within one of the circular chambers of the device, wherein the two populations of neurons may form synaptic connections with each other by means of extending axons through a series of one or more microchannels that connect the two chambers. By selecting the appropriate type(s) of neurons used to seed the two populations of neurons, and applying a suitable training regime, typically comprising the repetitive pairwise delivery of, for example, electrical stimuli to the [symbol] tion (e.g., neurons residing substantially within the left [symbol] d the “output” population (e.g., neurons residing substantially within the right-hand chamber), the neuronal gate may be trained to function as an NOT gate (i.e., a positive input signal applied to the input chamber results in suppression of signal (or in some cases a negative signal) at the output, and vice versa).

FIG. 1C provides a non-limiting example of a neuronal gate microstructure designed to support the growth of three populations of neurons (FIG. 1D), each population of neurons residing substantially within one of the circular chambers of the device, wherein the three populations of neurons may form synaptic connections with each other by means of extending axons through the microchannels that connect the two input chambers to the output chamber. By selecting the appropriate type(s) of neurons used to seed the two populations of neurons, and applying a suitable training regime as described previously, the neuronal gate may be trained to function as an AND gate (i.e., application of positive signals to both of the input chambers yields a positive response or signal at the output chamber) or an OR gate (i.e., application of a positive signal to at least one of the input chambers yields and positive response or signal at the output chamber).

### Example 2—A Biological Artificial Neural Network

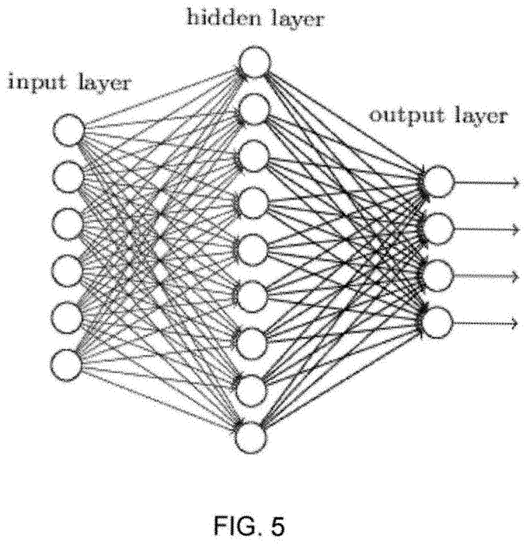

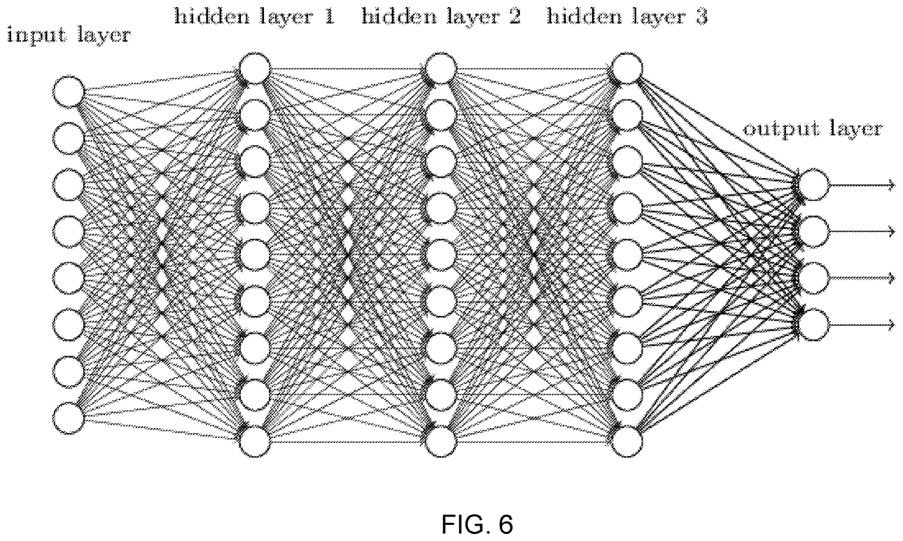

An artificial neural network (ANN) is one non-limiting example of a machine learning algorithm inspired by the structure and function of the human brain. Such algorithms (or models) generally comprise an interconnected group of nodes organized into multiple layers of nodes (FIG. 5). For example, the ANN architecture may comprise at least an input layer, one or more hidden layers, and an output layer. The ANN may comprise any total number of layers, and any number of hidden layers, where the hidden layers function as trainable feature extractors that allow mapping of a set of input data to an output value or set of output values. A so called “deep learning algorithm” is an ANN comprising a plurality of hidden layers, e.g., two or more hidden layers (FIG. 6).

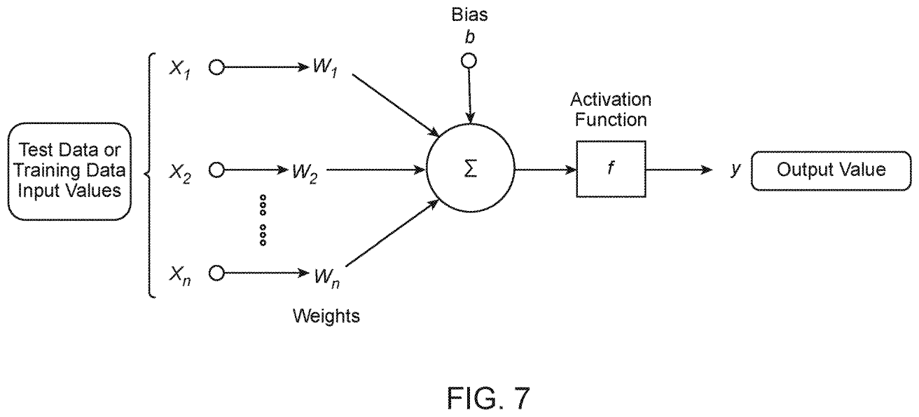

Each layer of the neural network comprises a number of nodes (or “neurons”). A node receives input that comes either directly from the input data (e.g., pixel position and intensity data in the case of image processing and feature recognition applications) or the output of nodes in previous layers, and performs a specific operation, e.g., a summation operation. In some cases, a connection from an input to a node is associated with a weight (or weighting factor). In some cases, the node may sum up the products of all pairs of inputs, xᵢ, and their associated weights (FIG. 7). In some cases, the weighted sum is offset with a bias, b. In some cases, the output of a node or neuron may be gated using a threshold or activation function, f, which may be a linear or non-linear function. The activation function may be, for example, a rectified linear uni [symbol] tion function, a Leaky ReLU activation function, or ar [symbol] ther functions known to those of skill in the art.

The weighting factors, bias values, and threshold values, or other computational parameters of the neural network, can be “taught” or “learned” in a training phase using one or more sets of training data. For example, the parameters may be trained using the input data from a training data set and a gradient descent or backward propagation method so that the output value(s) (e.g., a sample classification result) that the ANN computes are consistent with the examples included in the training data set.

In some cases, the disclosed biological processors or computers, which comprise two or more coupled neuronal gates, as illustrated in FIG. 4, may be configured and trained to function as the biological equivalent of an artificial neural network. For example, through the appropriate selection of the types of neurons used to seed the microwells or chambers of a microchip device comprising two or more coupled neuronal gates, and the application of the appropriate training regimen, the synaptic connectivity of the resulting network of neuronal gates may be trained to mimic the weighting factors, bias values, and threshold values of an artificial neural network that are “learned” during a training phase, such that the biological processor or computer may subsequently be used to process a specific combination of input signals and map the input to an appropriate set of output signals.

In one non-limiting example, the biological processors of the present disclosure may be trained using one or more training data sets that comprise single time point data or multi-time point (i.e., kinetic) data for the electrical signals (e.g., voltages or currents) recorded by one or more electrodes in one or more cell-based sensor devices such as those described in co-pending PCT Patent Application No. PCT/US2018/00027, along with the compound identities and concentrations of control samples to which the sensor devices have been exposed. Once trained, the biological processor or computer may then be used to map an input data set (e.g., a sensor signal pattern) to, for example, a determination of compound identity.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in any combination in practicing the invention. It is intended that the following claims define the scope of the invention and that methods and structures within the scope of these claims and their equivalents be covered thereby.

We claim:

1. A neuronal gate device comprising:
a) a microstructure comprising at least one input chamber and at least one output chamber, wherein the at least one input chamber is connected with the at least one output chamber through a series of two or more microchannels; and
b) at least two populations of neurons, wherein at least one population of neurons resides substantially within the at least one input chamber, and wherein at least one population of neurons resides substantially within the at least one output chamber;
wherein the populations of neurons within the at least one input chamber and the at least one output chamber are synaptically connected through the series of two or more microchannels.

2. The neuronal gate device of claim 1, wherein the device comprises a single input chamber connected to a single output chamber and functions as a NOT gate.

3. The neuronal gate device of claim 1, wherein the device comprises two input chambers and a single output chamber and functions as an AND gate.

4. The neuronal gate device of claim 1, wherein the device comprises two input chambers and a single output chamber and functions as an OR gate.

5. The neuronal gate device of claim 1, wherein the device further comprises a modulatory chamber connected with the at least one output chamber, wherein the modulatory chamber comprises a population of neurons that are synaptically connected with those in the output chamber and which function to modulate an output signal of the device.

6. The neuronal gate device of claim 1, wherein the microstructure is fabricated from glass, fused silica, silicon, photoresist, an elastomer, a polymer, or any combination thereof.

7. The neuronal gate device of claim 1, wherein the chambers have a maximum lateral dimension ranging from about 0.1 mm to about 2 mm.

8. The neuronal gate device of claim 1, wherein the chambers have a depth ranging from about 2 micrometers to about 15 micrometers.

9. The neuronal gate device of claim 1, wherein the synaptic connection between the population of neurons residing substantially within at least one input chamber and the population of neurons residing substantially within at least one output chamber is directional.

10. The neuronal gate device of claim 9, wherein the directional synaptic connection is implemented through the use of tapered microchannels to direct axon growth.

11. The neuronal gate device of claim 1, wherein the at least two populations of neurons comprise hippocampal neurons, cortical neurons, striatal neurons, or any combination thereof.

12. The neuronal gate device of claim 1, wherein the population of neurons residing substantially within at least one input chamber or output chamber is a homogeneous population of neurons, glial cells or astrocytes, Schwann cells, olfactory neuron cells, motor neurons, CNS neurons, Cortical neurons or any cells of neural origin.

13. The neuronal gate device of claim 1, wherein the population of neurons residing substantially within at least one input chamber or output chamber is a mixed population of neurons, glial cells or astrocytes, Schwann cells, olfactory neuron cells, motor neurons, CNS neurons, Cortical neurons or any cells of neural origin.

14. The neuronal gate device of claim 5, wherein the population of neurons residing substantially within the modulatory chamber comprises dopaminergic neurons.

15. The neuronal gate device of claim 14, wherein the dopaminergic neurons function to provide a feedback signal to minimize an error signal comprising the difference between an output signal of the device and a desired output signal.

16. The neuronal gate device of claim 1, wherein the synaptic connection between at least one input chamber and at least one output chamber is increased or decreased using a training regimen of applying paired stimuli to the neurons residing substantially within the input and output chambers in defined temporal patterns.

17. The neuronal gate device of claim 16, wherein a time delay between an input stimulus and an output stimulus of the paired stimuli used for training ranges from about −50 milliseconds to about +50 milliseconds.

18. The neuronal gate device of claim 16, wherein the paired stimuli are applied repetitively, and wherein a number of repetitions used for training ranges from about 10 to about 1; 000.

19. The neuronal gate device of claim 18, wherein a frequency of repetition ranges from about 0.1 Hz to about 100 Hz.

20. The neuronal gate device of claim 16, wherein the paired stimuli comprise optical signals, electrical signals, or any combination thereof.

21-54. (canceled)
